(19) United States
(12) Patent Application Publication
(10) Pub. No.: US 2004/0191785 A1
(43) Pub. Date: Sep. 30, 2004

(54) Methods for diagnosing individuals with an increased risk to develop a deficiency based on mdr1 gene polymorphism

(76) Inventors: Ulrich Brinkmann, Weilheim (DE); Michael Siegsmund, Heidelberg (DE)

Correspondence Address: James F Haley Jr, Fish & Neave, 1251 Avenue of the Americas, New York, NY, 10020-1104, US

(21) Appl. No.: 10/433,227

(22) PCT Filed: Nov. 29, 2000
(86) PCT No.: PCT/EP00/11974

(51) Int. Cl. C12Q 1/68

(52) U.S. Cl. 435/6

(57) ABSTRACT

The present invention relates to in vitro methods of diagnosing an increased risk to develop a renal, liver or colon deficiency, a deficiency in lymphoid cells or a deficiency at the blood brain barrier. Furthermore, the present invention also relates to the use of a MDR-1 gene single nucleotide polymorphism for the preparation of a diagnostic composition for diagnosing a renal, liver or colon deficiency, a deficiency in lymphoid cells or a deficiency at the blood brain barrier. Preferably, the renal, liver or colon deficiencies, the deficiency in lymphoid cells or the deficiencies at the blood brain barrier are caused by cancer.

The present invention relates to in vitro methods of diagnosing an increased risk to develop a renal, liver or colon deficiency, a deficiency in lymphoid cells or a deficiency at the blood brain barrier. Furthermore, the present invention also relates to the use of a MDR-1 gene single nucleotide polymorphism for the preparation of a diagnostic composition for diagnosing a renal, liver or colon deficiency, a deficiency in lymphoid cells or a deficiency at the blood brain barrier. Preferably, the renal, liver or colon deficiencies, the deficiency in lymphoid cells or the deficiencies at the blood brain barrier are caused by cancer.

Renal cell carcinoma (RCC) is the third most frequent urological tumor, accounting in the United States for 28,000 cases in the year 1995 (Wingo et al. 1995). About 11,000 patients die each year in the US from metastatic RCC (Wingo et al. 1995). This disease can only be cured if it is limited to the kidney, which allows surgical removal by radical nephrectomy. If the tumor has spread to distant organs, the prognosis is poor with 5 year survival rates of at most 20% (Guinan et al. 1995, Dinney et al. 1992). Therefore, to achieve high survival rates, it is necessary to detect the disease at an early stage, where it is curable by surgery. Ultrasound screening is a suitable and widespread, albeit relatively expensive and investigator dependent, method. Risk factors (e.g. mutations in the von Hippel-Lindau gene, familiar predisposition, or polycystic kidney disease Linehan et al. 1995, Schlehofer et al. 1996, Levine 1996) can be used to define risk groups that should be periodically examined by ultrasound.

The incidence of RCC has been increasing steadily by 2.3 to 4.3% per year in the United States and other industrialized countries in the Central and Northern regions of Europe depending upon race and gender (Chow et al. 1999). Wunderlich et al. (1999) showed that this increase can not be fully explained by the widespread use of ultrasound since the percentage of clinically recognized tumors on the total of all found RCCs in autopsy was nearly constant over a period of 12 years. The reason for this increase, especially in industrialized nations, has not yet been fully defined. However, a positive correlation between smoking, fat and meat consumption, obesity and hypertension with the occurence of RCC has been shown (Benichou et al. 1998, Schlehofer et al. 1996, Lindblad et al. 1997, Heath et al. 1997), whereas the intake of fruits, especially citrus fruits, vitamine C and E and carotene reduces the risk. It is feasible to assume that factors or genes that play a role in the defense of kidney cells against dietary and environmental toxins or metabolites may influence the individual susceptibility towards RCC.

The human multidrug resistance (MDR-1) gene is expressed in kidney cells in the proximal tubuli (Ambudkar et al. 1999, Gottesman et al. 1996) and its gene product, P-glycoprotein, is directly involved in the protection of cells against many toxic substances and metabolites. The P-glycoprotein is hereinafter also referred to as MDR-1 protein. MDR-1 encodes an integral membrane protein which pumps substances from the inside of cells and from membranes to the outside. The physiological role of this energy-dependent export mechanism is the protection of cells, although it may also play a role in steroid metabolism (Meda et al. 1987, Chen et al. 1990). MDR-1 is expressed in various organs, e.g. in the intestine bladder, prostate or in leucocytes as well as in the blood/brain barrier, where it controls the adsorption and penetration of substances (Schinkel et al. 1999, Rao et al. 1999). For example, in the kidney, located in proximal tubuli, it likely contributes to the efficient excretion of substances from the tubuli cells into the urine.

MDR-1 expression correlates directly with the “detoxification” capacity of cells in any of the above cited organs or tissues. This is of particular importance in cancer therapy, where high MDR-1 expression causes cancer cells to become refractory to treatment (Ambudkar et al. 1999). Likewise, it can be assumed that in non-malignant tissues or organs the degree of MDR-1 expression influences the capacity of the cells to remove damaging agents. Thus, the tubular cells of low expressors of MDR-1 are most likely more exposed to damaging substances compared to inherently high expressors.

Accordingly, means and methods for diagnosing an increased risk to develop one of the above deficiencies which are influenced either directly or indirectly by a cells ability of detoxification were hitherto not available but nevertheless highly desirable.

Thus, the technical problem underlying the present invention is to comply with the needs described above.

The solution to this technical problem is achieved by providing the embodiments characterized in the claims.

Accordingly, the present invention relates to an in vitro method of diagnosing an increased risk to develop a renal deficiency in a subject comprising

(a) determining the presence or absence of a single nucleotide polymorphism in the MDR-1 gene in a biological sample; and

(b) diagnosing a renal deficiency based on the presence of a single nucleotide polymorphism in the MDR-1 gene.

Furthermore, the present invention relates to an in vitro method of diagnosing an increased risk to develop a liver deficiency in a subject comprising

(a) determining the presence or absence of a single nucleotide polymorphism in the MDR-1 gene in a biological sample; and

(b) diagnosing a liver deficiency based on the presence of a single nucleotide polymorphism in the MDR-1 gene.

Moreover, the present invention relates to an in vitro method of diagnosing an increased risk to develop a colon deficiency in a subject comprising

(a) determining the presence or absence of a single nucleotide polymorphism in the MDR-1 gene in a biological sample; and

(b) diagnosing a colon deficiency based on the presence of a single nucleotide polymorphism in the MDR-1 gene.

In another embodiment the present invention relates to an in vitro method of diagnosing an increased risk to develop a deficiency in lymphoid cells in a subject comprising

(a) determining the presence or absence of a single nucleotide polymorphism in the MDR-1 gene in a biological sample; and

(b) diagnosing a colon deficiency based on the presence of a single nucleotide polymorphism in the MDR-1 gene.

The present invention also relates to an in vitro method of diagnosing an increased risk to develop a deficiency at the blood brain barrier in a subject comprising

(a) determining the presence or absence of a single nucleotide polymorphism in the MDR-1 gene in a biological sample; and

(b) diagnosing a colon deficiency based on the presence of a single nucleotide polymorphism in the MDR-1 gene.

By “an increased risk to develop” one of the deficiencies comprised by the above embodiments it is meant that some subjects may develop one of said deficiencies or a combination thereof statistically more likely than the average of a population comprising said subjects. Said increased risk may be caused by a variety of different risk factors such as genetic predispositions due to mutations in genes which are either directly responsible for the phenotypic consequences such as said deficiencies as well as genes which are indirectly responsible and effect cellular functions which are required to protect cells from damage by e.g. exogenous or endogenous toxic substances. Other sources of risk factors may be environmental factors such as frequent exposure of toxic substances or physiological factors such as stress and high workload. Usually, a combinatoin of said factors result in development of one of the above cited deficiencies. In particular, said deficiencies may be frequently observed in organs which have a high physiological activity characterized e.g. by a high level of metabolically active cells and/or a high level of proliferating cells such as liver cells, intestinal cells or lymphoid cells.

In addition, cells which are involved in tissue homeostasis and may therefore have barrier functions are also pivotal targets to develop said deficiencies. For example, said cells may be kidney cells or cells of the blood brain barrier.

In accordance with the above described embodiments of the present invention, said methods of diagnosing can be effected by determining the presence or absence of a single nucleotide polymorphism in the MDR-1 gene by standard molecular biology techniques well-known in the art. In principle, any suitable method for detecting a single nucleotide polymorphism is comprised by the present invention. Examples for said methods may comprise polynucleotide hybridization techniques such as Southern or Northern analysis or PCR-based techniques or techniques detecting altered physico-chemical properties such as mass spectroscopy. Depending on the nature of the biological sample in which the MDR-1 polynucleotide suspected to comprise said single nucleotide polymorphism has to be detected, the person skilled in the art may choose a suitable technique. For example, biological samples which comprise genomic DNA may be subjected to PCR techniques without further treatments while biological samples comprising RNA may be transcribed into DNA prior to applying PCR techniques, e.g., by reverse transcription of said RNA into DNA. All mentioned techniques are well known in the art, see e.g. Sambrook et al. (1989), Molecular Cloning: A Laboratory Manual., Cold Spring Harbor Laboratory Press, NY. The term “MDR-1 gene” as used herein refers to polynucleotides which may be either DNA or RNA or chemically, enzymatically or metabolically modified forms thereof. By “MDR-1 gene” it is meant that the gene may be either provided as genomic DNA or as transcribed RNA. In addition, also comprised by the invention are modified variants of said polynucleotides including, e.g., cDNA obtainable by reverse transcription of RNA encoding MDR-1.

Techniques of determining the presence or absence of a single nucleotide polymorphism are well known in the art and are described, e.g., in Sambrook et al. (1989), Molecular Cloning: A Laboratory Manual., Cold Spring Harbor Laboratory Press, NY. Said techniques may comprise visualization of the polynucleotide comprising the single nucleotide polymorphism directly or indirectly. For example, the polynucleotide or a fragment thereof comprising the single nucleotide polymorphism may be amplified prior to visualization by e.g. PCR techniques. Visualization may be accomplished by using e.g. labeled polynucleotide or oligonucleotide probes or by differences in the physico-chemical properties of said polynucleotide comprising a single nucleotide polymorphisms. The latter techniques may comprise, for instance, single-strand conformation polymorphism analysis (SSCP), restriction fragment length polymorphisms (RFLP) or mass spectroscopy.

For example, polynucleotide or oligonucleotide probes are preferably detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

One important parameter that had to be considered in the attempt to determine the individual MDR-1 genotype and identify novel MDR-1 variants by, e.g., direct DNA-sequencing of PCR-products from human blood genomic DNA is the fact that each human harbors (usually, with very few abnormal exceptions) two gene copies of each autosomal gene (diploidy). Because of that, great care had to be taken in the evaluation of the sequences to be able to identify unambiguously not only homozygous sequence variations but also heterozygous variations.

Advantageously, by providing the methods of the present invention it is now possible to identify subjects having a genetic predisposition and thus an increased risk to develop one of the above deficiencies based on low levels of MDR-1 gene expression or MDR-1 protein. Since, as discussed above, the development of said deficiencies might be dependent on further risk factors, said subjects may, thanks to the present invention, be efficiently subjected to means and measures of prevention. Moreover, by the methods provided by the present invention it might be possible to reduce side effects of medical therapies, such as chemotherapy in subjects comprising said single nucleotide polymorphisms. Said side effects may be caused by an insufficient detoxification capacity of the cells of a subject resulting e.g. in accumulation of therapeutic agents in cells of said subject up to harmful levels. Said subjects can be efficiently and easily identified and subsequently subjected to alternative and may be less harmful therapies resulting in less side effects.

The definitions used herein above for the other embodiments of the invention also apply for the embodiments described hereinafter.

In a preferred embodiment the method of the invention is comprising PCR, ligand string reactions, restriction digestion, direct sequencing, nucleic acid amplification techniques, hybridization techniques, immunoassays or mass spectroscopy.

In another embodiment the present invention relates to an in vitro method of diagnosing an increased risk to develop a renal deficiency in a subject comprising

(a) determining the presence or absence of the MDR-1 polypeptide in a biological sample; and

(b) diagnosing a renal deficiency based on the absence of the MDR-1 polypeptide.

The present invention also relates to an in vitro method of diagnosing an increased risk to develop a liver deficiency in a subject comprising

(a) determining the presence or absence of the MDR-1 polypeptide in a biological sample; and

(b) diagnosing a liver deficiency based on the absence of the MDR-1 polypeptide.

Further, the present invention relates to an in vitro method of diagnosing an increased risk to develop a colon deficiency in a subject comprising

(a) determining the presence or absence of the MDR-1 polypeptide in a biological sample; and

(b) diagnosing a colon deficiency based on the absence of the MDR-1 polypeptide.

In yet another embodiment the present invention relates to an in vitro method of diagnosing an increased risk to develop deficiency in lymphoid cells in a subject comprising

(a) determining the presence or absence of the MDR-1 polypeptide in a biological sample; and

(b) diagnosing a colon deficiency based on the absence of the MDR-1 polypeptide.

In still another embodiment the present invention relates to an in vitro method of diagnosing an increased risk to develop deficiency at the blood brain barrier in a subject comprising

(a) determining the presence or absence of the MDR-1 polypeptide in a biological sample; and

(b) diagnosing a colon deficiency based on the absence of the MDR-1 polypeptide.

In accordance with the present invention determining the presence or absence of the MDR-1 polypeptide may be carried out by any available standard molecular biology technique known in the art. For example, said techniques may comprise immunoassays, antibody detection techniques, such as Western blotting, histological techniques comprising immuno-histological and enzyme-histological methods, FACS analysis, enzymatic techniques or other suitable techniques which detect the MDR-1 protein. Preferably, antibodies employed in such techniques may be antibodies such as a monoclonal antibody, a polyclonal antibody, a single chain antibody, human or humanized antibody, primatized, chimerized or fragment thereof that specifically binds said peptide or polypeptide also including bispecific antibody, synthetic antibody, antibody fragment, such as Fab, Fv or scFv fragments etc., or a chemically modified derivative of any of these. The general methodology for producing antibodies is well-known and has been described in, for example, Kohler and Milstein, Nature 256 (1975), 494 and reviewed in J. G. R. Hurrel, ed., “Monoclonal Hybridoma Antibodies: Techniques and Applications”, CRC Press Inc., Boco Raron, Fla. (1982), as well as that taught by L. T. Mimms et al., Virology 176 (1990), 604-619. Furthermore, antibodies or fragments thereof to the aforementioned peptides can be obtained by using methods which are—described, e.g., in Harlow and Lane “Antibodies, A Laboratory Manual”, CSH Press, Cold Spring Harbor, 1988. Said techniques may be either applied to a biological sample comprising a plurality of compounds or said sample may be first subjected to a selection process, e.g., protein purification techniques. Said further techniques which may be applied prior to the above mentioned techniques are also well known in the art.

Visualization techniques which are suitable for determination of the MDR-1 protein are dependent on the above described methods and are also comprised by the methods of the invention referred to herein above. Polypeptides, such as antibodies or other polypeptides capable of binding to MDR-1 are preferably detectably labeled. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, RH and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. There are many different labels and methods of labeling known to those of ordinary skill in the art. Commonly used labels comprise, inter alia, fluorochromes (like fluorescein, rhodamine, Texas Red, etc.), enzymes (like horse radish peroxidase, β-galactosidase, alkaline phosphatase), radioactive isotopes (like ³²P or ¹²⁵I), biotin, digoxygenin, colloidal metals, chemi- or bioluminescent compounds (like dioxetanes, luminol or acridiniums). Labeling procedures, like covalent coupling of enzymes or biotinyl groups, iodinations, phosphorylations, biotinylations, random priming, nick-translations, tailing (using terminal transferases) are well known in the art. Detection methods comprise, but are not limited to, autoradiography, fluorescence microscopy, direct and indirect enzymatic reactions, etc.

As discussed above, the present invention allows, based on the correlation between single nucleotide polymorphisms carrying low level MDR-1 expression, the efficient and reliable identification of a risk factor for the development of the above-described deficiencies.

The identification of a single nucleotide polymorphism in accordance with the present invention has to be seen as identification of a key risk factor, since the susceptibility of a subject to other risk factors may increase due to low level MDR-1 expression. In particular, toxic compounds may accumulate in the cells of said subject due to the lack of ability of detoxification. Organs or tissues which are usually effected by said lack of ability of detoxification are the organs or tissues which develop the above-mentioned deficiencies. Said organs or tissues are all essential for the physiology of a subject such as a human being. The provision of the methods of the present invention allows efficient prevention of said deficiencies which can unfortunately most often only be treated by complicated and harmful chemotherapy or organ transplantation.

In a preferred embodiment the method of the present invention is comprising immunoassays, antibody detection techniques, histological techniques, FACS analysis, enzymatic techniques, techniques which detect the MDR-1 protein.

In another preferred embodiment of the method of the present invention said renal deficiency is caused by cancer.

The term “cancer” as used herein and herein after comprises cancer which may be derived from all cell types present in the deficient organ. Said cell types may be epithelial cells, mesenchynal cells, endothelial cells or specialized cells of said organs. Thus, the cancer may be classified, e.g., as a carcinoma, sarkoma, hemangioma or leukemia. Phenotypic and physiological characteristics of cancer diseases are well known in the art and are described in standard text books such as Pschyrembel.

In a most preferred embodiment of the method of the present invention said cancer is renal cell carcinoma (RCC).

In a preferred embodiment of the method of the present invention said liver deficiency is caused by cancer.

In another preferred embodiment of the method of the present invention said cancer is liver cancer.

In a further preferred embodiment of the method of the present invention said colon deficiency is caused by cancer.

In a particularly preferred embodiment of the method of the present invention said cancer is colon cancer.

In a preferred embodiment of the method of the present invention said deficiency in lymphoid cells is caused by cancer.

In a most preferred embodiment of the method of the present invention said cancer is leukemia.

In another most preferred embodiment of the method of the present invention said deficiency in lymphoid cells is caused by cancer.

In a particularly preferred embodiment of the method of the present invention said cancer is a hemangioma.

The present invention also relates to the use of a MDR-1 gene single nucleotide polymorphism for the preparation of a diagnostic composition for diagnosing an increased risk to develop a renal deficiency.

The definitions used herein above for the terms used in the embodiments referring to methods provided by the present invention can be applied mutatis mutandis to the use referred to herein above and the uses referred to herein after.

The diagnostic composition of the present invention may contain further ingredients suitable for diagnosing the deficiencies referred to in the embodiments above and hereinafter. A diagnostic composition in accordance with the invention to be used for detection of a polynucleotide comprising a single nucleotide polymorphism or a polypeptide or protein can also be applied in vivo for imaging. For example, antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

Said diagnostic composition may be comprised by a kit. The kit of the invention may advantageously be used for carrying out a method or use of the invention and could be, inter alia, employed in a variety of applications, e.g., in the diagnostic field or as research tool. The parts of the kit of the invention can be packaged individually in vials or in combination in containers or multicontainer units. Manufacture of the kit follows preferably standard procedures which are known to the person skilled in the art. For example, the kit or diagnostic compositions may be used for methods or uses for detecting expression of a MDR-1 gene suspected to comprise a single nucleotide polymorphism in accordance with any one of the above-described methods of the invention, employing, for example, immuno assay techniques such as radioimmunoassay or enzymeimmunoassay or preferably nucleic acid hybridization and/or amplification techniques such as those described herein before and in the examples.

In a preferred embodiment of the use of the invention said renal deficiency is caused by cancer.

In another preferred embodiment of the use of the present invention said cancer is renal cell carcinoma (RCC).

The present invention also relates to the use of a MDR-1 gene single nucleotide polymorphism for the preparation of a diagnostic composition for diagnosing an increased risk to develop a liver deficiency.

In a preferred embodiment of the use of the present invention said liver deficiency is caused by cancer.

In a further preferred embodiment of the use of the present invention said cancer is liner cancer.

Furthermore, the present invention relates to the use of a MDR-1 gene single nucleotide polymorphism for the preparation of a diagnostic composition for diagnosing an increased risk to develop a colon deficiency.

In a preferred embodiment of the use of the present invention said colon deficiency is caused by cancer.

In another preferred embodiment of the use of the present invention said cancer is colon cancer.

The present invention also relates to the use of a MDR-1 gene single nucleotide polymorphism for the preparation of a diagnostic composition for diagnosing an increased risk to develop a deficiency in lymphoid cells.

In a preferred embodiment of the use of the present invention said deficiency in lymphoid cells is caused by cancer.

In a further preferred embodiment of the use of the present invention said cancer is leukemia.

Furthermore, the present invention relates to the use of a MDR-1 gene single nucleotide polymorphism for the preparation of a diagnostic composition for diagnosing an increased risk to develop a deficiency at the blood brain barrier.

In a preferred embodiment of the use of the present invention said deficiency at the blood brain barrier is caused by cancer.

Moreover, in a preferred embodiment of the use of the present invention said cancer is a hemangioma.

In a preferred embodiment of the method of the present invention the single nucleotide polymorphism is

(a) a nucleotide substitution, addition, deletion or substitution and addition at a position corresponding to position 3435 of exon 26 of the MDR-1 gene (Accession No: AF016535);

(b) a C to T substitution at a position corresponding to position 3435 of exon 26 of the MDR-1 gene (Accession No: AF016535); or

(c) a C to T substitution at position 3435 of exon 26 of the MDR-1 gene (Accession No: AF016535).

The MDR-1 C3435T polymorphism, which influences intestinal expression and uptake of MDR-1 substrates, also correlates with renal expression of MDR-1 protein. For example, individuals homozygous for the T-allele display significantly lower expression of MDR-1 in the proximal tubuli of the kidney compared to heterozygous and homozygous C-allele carriers (p=0.06, N=19CC and 28TT). Since MDR-1 protein plays a role in the excretion of toxic substances, the tubular cells of individuals with homozygous low expressor (T) alleles are more exposed to toxic agents. In agreement with the hypothesis that exposure to noxic or toxic agents promotes the development of deficiencies such as renal cell carcinoma (RCC), it was found that the MDR-1 3435T allele is a susceptibility factor for e.g. RCC: Individuals that are homozygous T-allele carriers (24% of the normal population, N>500 but 36% in RCC, N=222) have an significantly increased risk to develop deficiencies such as RCC compared to heterozygous or homozygous carriers of the C-allele. The odds ration (OR) for developing RCC due to the MDR-1 polymorphic genotype (T/T) was <1.5 for all age groups (p=0.028) and OR=2.1 for patients older than 50 yrs (p=0.001). 36% of RCC patients carried homozygously the T-allele compared to 26% in the control population, while only 18% of RCC patients carried homozygously the high expressor C-allele compared to 25% in the controls. This difference was even more pronounced when RCC patients were distinguished in those with (n=45) and without (n=76) somatic VHL mutations. 36% of the patients with VHL mutations carried homozygously the T-allele, a 40% increase compared to the normal allelic distribution. Our results indicate that genetically determined variations in renal MDR-1 expression play a significant role in RCC, contributing to 15% of all RCC (Ef=0.15).

Further applications of the polymorphisms identified in accordance with the present invention and means and methods that can be used in accordance with the above described embodiments can be found in the prior art, for example, as described in U.S. Pat. No. 5,856,104, wherein the there described means and methods for forensics, Paternity testing, correlation of polymorphisms with phenotypic traits, genetic mapping of phenotypic traits, etc. can be equally applied in accordance with the present invention.

These and other embodiments are disclosed or are obvious from and encompassed by the description and examples of the present invention. Further literature concerning any one of the methods, uses and compounds to be employed in accordance with the present invention may be retrieved from public libraries, using for example electronic devices. For example the public database “Medline” may be utilized which is available on Internet, e.g. under http://www.ncbi.nlm.nih.gov/PubMed/medline.html. Further databases and addresses, such as http://www.ncbi.nlm.nih.gov/, http://www.infobiogen.fr/, http://www.fmi.ch/biology/research_tools.html, http://www.tigr.org/, are known to the person skilled in the art and can also be obtained using, e.g., http://www.lycos.com. An overview of patent information in biotechnology and a survey of relevant sources of patent information useful for retrospective searching and for current awareness is given in Berks, TIBTECH 12 (1994), 352-364.

The compositions, methods and uses of the present invention may be desirably employed in humans, although animal treatment is also encompassed by the methods and uses described herein.

The invention is illustrated by the figures:

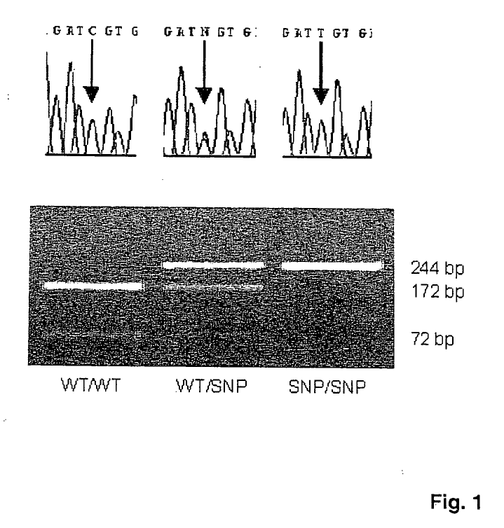

FIG. 1: Detection of MDR-1 polymorphisms

Identification of homozygous and heterozygous carriers of the C3435T polymorphism by PCR-RFLP and sequence analysis. The polymorphism can be detected directly in the DNA sequence profiles, or by restriction digestion of the 244 bp PCR fragments with Sau3 μl or Mbol, which cleaves the C-allele to 172 bp and 72 bp, but not the T-allele.

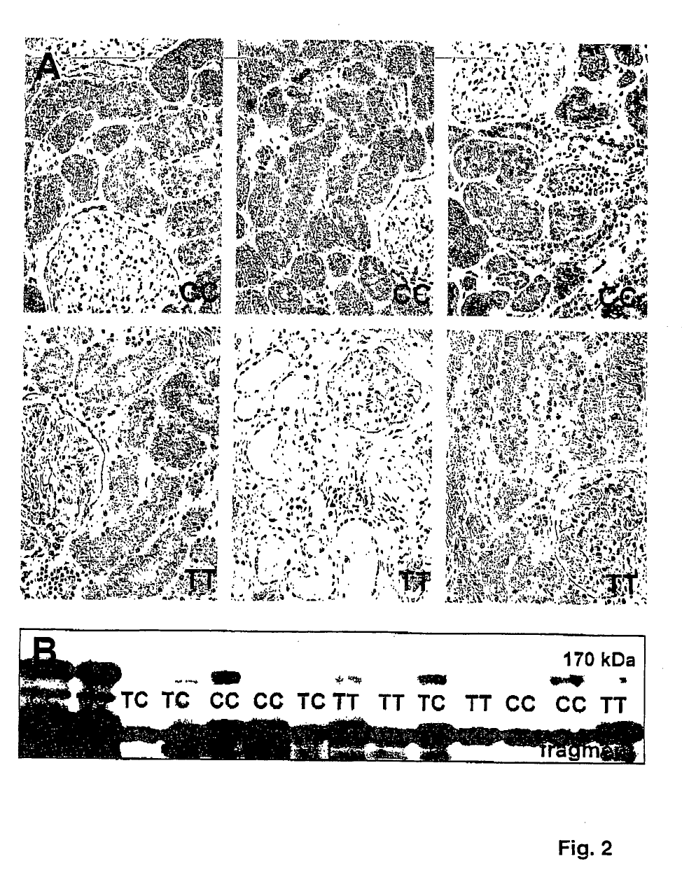

FIG. 2: Variable expression of PGP in the kidney

Immunohistology and Western blots with anti-PGP antibody JSB-1 with standardized conditions, shows large variation in tubular PGP levels. (A) Immunohistochemical staining, the MDR-1 alleles of the samples are indicated. (B) Western blots show PGP specific staining at 170 kDa as well as bands (degradation products) of smaller sizes. Note that the intensity of full length protein as well as smaller fragments is generally stronger in C-C samples compared to samples with the T-T genotype.

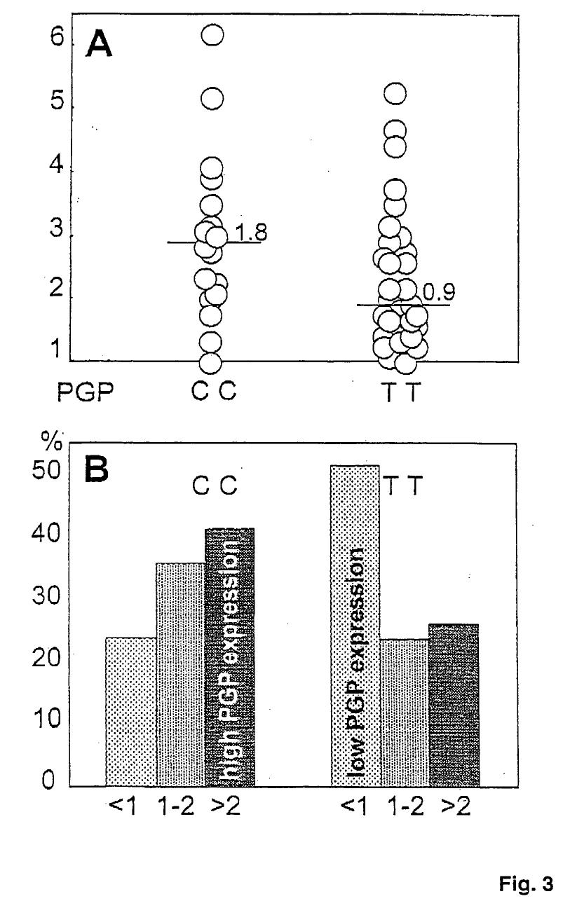

FIG. 3: The C3435T polymorphism correlates with renal MDR-1 expression

Individual PGP levels were obtained by Histoanalyzer analyses of stained tissue slides from 18 homozygous C-allele carriers and 31 homozygous T-allele carrying individuals (see FIG. 2, Methods). (A) Individual signal to background ratios: lower expressing individuals are mostly found in the T-allele group with relative PGP signals between 1 and 2. A ratio of 1.0 is no specific staining above background. (B) Genotype-phenotype correlation after clustering the histological data in 3 groups (high, medium and low expressors) The majority of the C-C individuals display high renal PGP levels, while most T-individuals have low PGP levels.

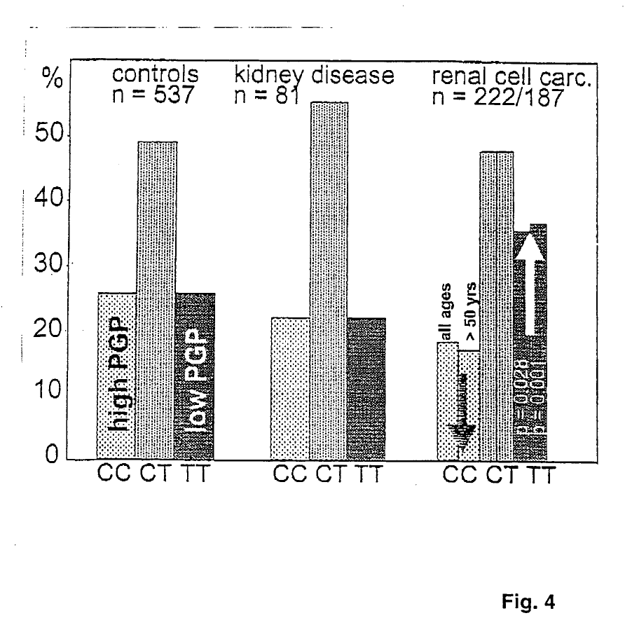

FIG. 4: The 3435T polymorphism in renal cell carcinoma

The distribution of the C3435T polymorphism in Caucasians has been analyzed in more that 500 controls, 81 samples with benign end-stage kidney disease and 222 RCC patients. The controls and the benign kidney disease patients show a “normal” allelic distribution with 22-26% 3435T homozygotes and a distribution that fits the Hardy Weinberg equlilibrium. The RCC population carries significantly more (36%, p=0.001-0.028, Fischer Test) homozygous carriers of the T allele.

The invention will now be described by reference to the following biological examples which are merely illustrative and are not to be constructed as a limitation of scope of the present invention.

### EXAMPLE 1

DNA Samples and Genotyping

Tumor and normal renal tissue samples from 222 caucasian RCC patients were obtained from the tumor collection of the Department of Urology at the University Hospital Mannheim and from the Dr. Margarete Fischer Bosch Institute for Clinical Pharmacology in Stuttgart, and blood samples from 81 patients with benign end-stage kidney disease from the Dialysis Center Donauworth (Table 1). Control samples of individuals with no known kidney disease or tumors, for which the C3435T polymorphism had been determined, were from the Epidauros DNA collection. Genomic DNA was prepared from tissue and blood samples using the Qiagen (QiaAmp) kits. The primers 5′GATCTGTGAACTCTTGTTTTCA3′ (SEQ ID NO:1) and 5′GMGAGA GACTTACATTAGGC3′ (SEQ ID NO:2) were used for PCR of the exon 26 MDR-1 gene fragment. In a volume of 20 μl, 20 ng genomic DNA was added to buffer containing 1.5 mM MgCl2, 250 μM dNTP′, 20 pmol of each primer and 1 U Taq polymerase. PCR was carried out with initial denaturation of 3 min at 95° C. followed by 30 cycles of 94° C. for 30 sec, 30 sec at 62° C., and 30 sec at 72° C. The C3435T polymorphism was detected by RFLP with Sau3AI or Mbol (FIG. 1), or by direct sequencing ABI3700 sequencers using BigDye Terminators in cases (some heterozygotes) which required genotype confirmation.

### EXAMPLE 2

Variations of MDR-1 Expression in the Kidney

The expression of MDR-1 in normal tissues can show large interindividual differences (e.g. in the intestine, Greiner et al. 1999) To measure the degree of individual variability of renal expression, we determined the PGP levels in kidney samples from patients from the Department of Urology at the University Hospital Mannheim. All patients suffered renal cell carcinoma (see table 1 for details). Tumor samples as well as corresponding healthy renal tissue (from tumor surgery) was obtained and stored as frozen tissue blocks. For analyses of the levels of PGP by Western blots and quantitative immunohistology, only the normal non-cancerous tissues were used. Microscopic inspection of these tissue sections showed normal healthy morphology and no indication of malignancy. The analysis of PGP levels in these samples (FIG. 2) showed high interindividual variability of PGP expression. By immunohistology, PGP was found to be specifically expressed in many samples in significant amounts in the proximal tubuli. In contrast, other samples showed weak expression and in some the PGP stain was just barely visible. Quantitative measurements (Histoanalyzer) of the staining intensities demonstrated up to 6 fold differences in individual PGP expression (FIG. 3). This interindividual variability could also be confirmed by Western blot analyses (FIG. 2B). PGP levels were analyzed by Western Blot and quantitative immunohistochemistry as described for intestinal quantification (Greiner et al. 1999), using the mouse monoclonal antibody JSB-1 (Roche, 50 ug/ml) diluted to 5 ug/ml. Histological slides were stained with DAB (Vectastain ABC complex) and counterstained with HE (Merck). The PGP-specific staining was analyzed with a histoanalyzer (Greiner et al. 1999), using ratios of specific signals (tubuli) to background to determine individual PGP expression.

### EXAMPLE 3

The C3435T Polymorphism Correlates with Renal MDR-1 Expression

To analyze whether the C3435T polymorphisms, which is linked to low PGP expression and function in the intestine, also correlates with the variable PGP levels in the kidney, we genotyped the RCC patients (Table 1). The MDR-1 C3435T genotype was determined from genomic DNA isolated from tumor as well as the corresponding healthy renal tissue of these patients. No genotype differences, i.e no gene conversions were found between the sets of healthy and cancerous samples. For the correlation of MDR-1 genotype and PGP expression in the kidney, quantitative immunohistochemistry was performed for individuals that were homozygous for the C3435T polymorphism (19 CC, 28 TT). The results of this analysis. (FIG. 3) show a strong correlation of MDR-1 genotype and renal PGP expression (p=0.06, Kruskal-Wallis Test, p=0.039 Median Test). The group which carries homozygously the C-allele shows higher levels of PGP compared to individuals with the corresponding T-allele. A comparison of the readouts from the histoanalyzer (FIG. 3A) shows that the lower expressing individuals are mostly found in the T-allele group (relative PGP signals between 1 and 2), the highest value comes from an individual with homozygous C-alleles (PGP signal >6). The difference in expression levels is approximately 2-fold (mean values 0.91, TT vs 1.77, CC above the non-specific signal ratio 1.0). Considering the scatter that can occur in immunohistological experiments, we have also analyzed the genotype distribution after clustering the histological data in just 3 groups (high with signal strength ratios >3, medium 2-3, and low expressing individuals signal strength <2, FIG. 3B). Correlations of the MDR-1 genotypes with these expression groups show that the majority of the C/C individuals display high renal PGP levels, while the majority of homozygous T-allele individuals have low PGP levels.

### EXAMPLE 4

The C3435T Low Expresser Polymorphism is a Risk Factor for Renal Cell Carcinoma

Large numbers of Caucasians have been genotyped for the C3435T polymorphism that correlates with intestinal PGP concentration and PGP dependent drug transport, to obtain reliable information about the distribution of this allele. Previous studies as well as ongoing experiments show consistantly that in normal Caucasians this polymorphism is present homozygously in 22-26% of the population and heterozygous in 48-50% (Hoffineyer et al. 2000). This distribution, which fits the Hardy Weinberg equlilibrium of alleles in a population, has been confirmed in more than 500 samples. In contrast to that, the frequencies of homozygous and heterozygous C3435T individuals of the RCC population show a different distribution. In this population, the frequency of homozygous carriers of the T-allele is significantly increased from 26% (normal population) to 35 to 39% (FIG. 4). Genotyping of additional 57 patients with benign end-stage kidney disease (defined by requirement to undergo regular dialysis, see table 1) as control group indicated no increased frequency of the T-allele and no allelic disequilibrium in this group. The RCC patient group comprises a notable size (N=222), and the difference in allelic frequences is statistically significant. The allelic distribution found in the RCC group also differs from the Hardy Weinberg equilibrium, with homozygous T allele carriers clearly being enriched in RCC (FIG. 4). The statistical evaluation (ANOVA) of these allelic differences show that the MDR-1 C3435T allele comprizes a significant risk factor, with homozygous T-allele carriers having a 2-fold increased probability to develop RCC (p=0.001, Fischer Test), in the age groups >50 years, which is the most predominant population in which RCC occurs. Statistical analysis was performed using SPSS 10.0 (SPSS, Chicago, USA). The statistical analysis of pGP expression in renal tissue was primarily based on non-parametric methods. The median test was used to compare pGP expression in MDR1 tt samples versus cc or ct genotype. The odds ratio approximation (OR) and Cl were used to analyse the frequency distribution of genotypes in patients and controls and the Pearson chi-square was calclated to test the equality of proportions.

The etiological fraction was calculated as

$${E}_{f} = {P}_{e} \cdot \frac{( OR - 1 )}{{P}_{e} \cdot ( OR - 1 ) + 1} ,$$

were Pₑ is the proportion of subjects with the susceptibility genotype (tt).

TABLE 1

| | | total | MDR1 C > T₃₄₃₅ | | | OR | |
|---|---|---|---|---|---|---|---|
| | | n | cc | ct | tt | (95% Cl) | p¹ |
| healthy controls | n | 537 | 139 | 256 | 142 | | |
| | % | | 25.9 | 47.7 | 26.4 | | |
| benign kidney disease | | | | | | | |
| glomerulonephritis | n | 11 | 1 | 7 | 3 | | |
| | % | | 9.1 | 63.8 | 27.3 | | |
| IDDM | n | 16 | 6 | 9 | 1 | | |
| | % | | 37.5 | 56.3 | 6.2 | | |
| AKPD | n | 6 | 1 | 3 | 2 | | |
| | % | | 16.7 | 50.0 | 33.3 | | |
| mPAN | n | 2 | | 2 | | | |
| | % | | | 100.0 | | | |
| IGA nephritis | n | 5 | 1 | 3 | 1 | | |
| | % | | 20.0 | 60.0 | 20.0 | | |
| unknown | n | 41 | 9 | 21 | 11 | | |
| | % | | 22.0 | 51.2 | 26.8 | | |
| age | | | | | | | |
| ≦50 yrs. | n | 20 | 4 | 14 | 2 | | |
| | % | | 20.0 | 70.0 | 10.0 | | |
| >50 yrs. | n | 61 | 14 | 31 | 16 | | |
| | % | | 23.0 | 50.8 | 26.2 | | |
| total | n | 81 | 18 | 45 | 18 | 0.77 | 0.467 |
| | % | | 22.2 | 65.8 | 22.2 | (0.39-1.44) | |
| Renal cell carcinoma | | | | | | | |
| histology | | | | | | | |
| clear cell | n | 188 | 33 | 94 | 61 | | |
| | % | | 17.6 | 50.0 | 32.5 | | |
| other | n | 34 | 7 | 11 | 16 | | |
| | % | | 20.6 | 32.4 | 47.1 | | |
| gender | | | | | | | |
| female | n | 106 | 31 | 47 | 28 | | |
| | % | | 29.2 | 44.3 | 26.4 | | |
| male | n | 194 | 49 | 93 | 52 | | |
| | % | | 25.3 | 47.9 | 26.8 | | |
| age | | | | | | | |
| ≦50 yrs. | n | 25 | 6 | 12 | 7 | | |
| | % | | 24.0 | 48.0 | 28.0 | | |
| >50 yrs. | n | 187 | 33 | 88 | 66 | 2.09 | <0.001 |
| | % | | 17.6 | 47.1 | 35.3 | (1.41-3.08) | |
| total | n | 222 | 40 | 105 | 77 | 1.48 | 0.028 |
| | % | | 18.0 | 47.3 | 34.7 | (1.04-2.09) | |

¹calculated versus healthy controls considering MDR1 TT as risk factor.

Table 1: Patient groups and MDR1 genotypes:

IDDM, insulin dependent diabetes melitus, APKD, adult polycystic kidney disease. The unknown group is diagnosed by the requirement to undergo regular dialysis, without any further knowledge about the underlying disease (not IDDM).

TABLE 2

| Samples | N | % CC | % CT | % TT |
|---|---|---|---|---|
| Controls | 537 | 25.9 | 47.7 | 26.4 |
| Benign Kidney Disease | 81 | 22.2 | 55.6 | 22.2 |
| CCRCC, wt VHL | 76 | 11.8 | 57.9 | 30.3 |
| CCRCC, mut VHL | 45 | 13.1 | 51.1 | 35.6 |

Table 2: MDR C₃₄₃₅T genotype and VHL mutations

1. An in vitro method of diagnosing an increased risk to develop a renal deficiency in a subject comprising
(a) determining the presence or absence of a single nucleotide polymorphism in the MDR-1 gene in a biological sample; and
(b) diagnosing a renal deficiency based on the presence of a single nucleotide polymorphism in the MDR-1 gene.

2. An in vitro method of diagnosing an increased risk to develop a liver deficiency in a subject comprising
(a) determining the presence or absence of a single nucleotide polymorphism in the MDR-1 gene in a biological sample; and
(b) diagnosing a liver deficiency based on the presence of a single nucleotide polymorphism in the MDR-1 gene.

3. An in vitro method of diagnosing an increased risk to develop a colon deficiency in a subject comprising
(a) determining the presence or absence of a single nucleotide polymorphism in the MDR-1 gene in a biological sample; and
(b) diagnosing a colon deficiency based on the presence of a single nucleotide polymorphism in the MDR-1 gene.

4. An in vitro method of diagnosing an increased risk to develop a deficiency in lymphoid cells in a subject comprising
(a) determining the presence or absence of a single nucleotide polymorphism in the MDR-1 gene in a biological sample; and
(b) diagnosing a colon deficiency based on the presence of a single nucleotide polymorphism in the MDR-1 gene.

5. An in vitro method of diagnosing an increased risk to develop a deficiency at the blood brain barrier in a subject comprising
(a) determining the presence or absence of a single nucleotide polymorphism in the MDR-1 gene in a biological sample; and
(b) diagnosing a colon deficiency based on the presence of a single nucleotide polymorphism in the MDR-1 gene.

6. The method of any one of claims 1 to 5 comprising PCR, ligand string reactions, restriction digestion, direct sequencing, nucleic acid amplification techniques, hybridization techniques, immunoassays or mass spectroscopy.

7. An in vitro method of diagnosing an increased risk to develop a renal deficiency in a subject comprising
(a) determining the presence or absence of the MDR-1 polypeptide in a biological sample; and
(b) diagnosing a renal deficiency based on the absence of the MDR-1 polypeptide.

8. An in vitro method of diagnosing an increased risk to develop a liver deficiency in a subject comprising
(a) determining the presence or absence of the MDR-1 polypeptide in a biological sample; and
(b) diagnosing a liver deficiency based on the absence of the MDR-1 polypeptide.

9. An in vitro method of diagnosing an increased risk to develop a colon deficiency in a subject comprising
(a) determining the presence or absence of the MDR-1 polypeptide in a biological sample; and
(b) diagnosing a colon deficiency based on the absence of the MDR-1 polypeptide.

10. An in vitro method of diagnosing an increased risk to develop deficiency in lymphoid cells in a subject comprising
(a) determining the presence or absence of the MDR-1 polypeptide in a biological sample; and
(b) diagnosing a colon deficiency based on the absence of the MDR-1 polypeptide.

11. An in vitro method of diagnosing an increased risk to develop deficiency at the blood brain barrier in a subject comprising
(a) determining the presence or absence of the MDR-1 polypeptide in a biological sample; and
(b) diagnosing a colon deficiency based on the absence of the MDR-1 polypeptide.

12. The method of any one of claims 7 to 11 comprising immunoassays, antibody detection techniques, histological techniques, FACS analysis, enzymatic techniques, techniques which detect the MDR-1 protein.

13. The method of claim 1 or 7, wherein said renal deficiency is caused by cancer.

14. The method of claim 13, wherein said cancer is renal cell carcinoma (RCC).

15. The method of claim 2 or 8, wherein said liver deficiency is caused by cancer.

16. The method of claim 11, wherein said cancer is liver cancer.

17. The method of claim 3 or 9, wherein said colon deficiency is caused by cancer.

18. The method of claim 13, wherein said cancer is colon cancer.

19. The method of claim 4 or 10, wherein said deficiency in lymphoid cells is caused by cancer.

20. The method of Claim 19, wherein said cancer is leukemia.

21. The method of claim 5 or 11, wherein said deficiency in lymphoid cells is caused by cancer.

22. The method of claim 21 wherein said cancer is a hemangioma.

23. Use of a MDR-1 gene single nucleotide polymorphism for the preparation of a diagnostic composition for diagnosing an increased risk to develop a renal deficiency.

24. The use of claim 23, wherein said renal deficiency is caused by cancer.

25. The use of claim 24, wherein said cancer is renal cell carcinoma (RCC).

26. Use of a MDR-1 gene single nucleotide polymorphism for the preparation of a diagnostic composition for diagnosing an increased risk to develop a liver deficiency.

27. The use of claim 26, wherein said liver deficiency is caused by cancer.

28. The use of claim 27, wherein said cancer is liver cancer.

29. Use of a MDR-1 gene single nucleotide polymorphism for the preparation of a diagnostic composition for diagnosing an increased risk to develop a colon deficiency.

30. The use of claim 29, wherein said colon deficiency is caused by cancer.

31. The use of claim 30, wherein said cancer is colon cancer.

32. Use of a MDR-1 gene single nucleotide polymorphism for the preparation of a diagnostic composition for diagnosing an increased risk to develop a deficiency in lymphoid cells.

33. The use of claim 32, wherein said deficiency in lymphoid cells is caused by cancer.

34. The use of claim 33, wherein said cancer is leukemia.

35. Use of a MDR-1 gene single nucleotide polymorphism for the preparation of a diagnostic composition for diagnosing an increased risk to develop a deficiency at the blood brain barrier.

36. The use of claim. 35, wherein said deficiency at the blood brain barrier is caused by cancer.

37. The use of claim 36, wherein said cancer is a hemangioma.

38. The method of any one of claims 1 to 22 or the use of any one of claims 23 to 37, wherein the single nucleotide polymorphism is
(a) a nucleotide substitution, addition, deletion or substitution and addition at a position corresponding to position 3435 of exon 26 of the MDR-1 gene (Accession No: AF016535);
(b) a C to T substitution at a position corresponding to position 3435 of exon 26 of the MDR-1 gene (Accession No: AF016535); or
(c) a C to T substitution at position 3435 of exon 26 of the MDR-1 gene (Accession No: AF016535).


SEQUENCE LISTING (2 sequences)

SEQ ID NO: 1 - DNA, 22 bp, Homo sapiens
  misc_feature: primer sequence for amplification of exon 26 of MDR1
gatctgtgaactcttgttttca

SEQ ID NO: 2 - DNA, 21 bp, Homo sapiens
  misc_feature: primer sequence for amplification of exon 26 of MDR1
gaagagagacttacattaggc